(19) United States
(12) Patent Application Publication
(10) Pub. No.: US 2026/0271980 A1
(43) Pub. Date: Sep. 17, 2026

(54) PARTICULATE NUTRITIONAL COMPOSITION

(71) Applicant: N.V. Nutricia, Zoetermeer (NL)

(72) Inventors: Martijn Johannes van der Hoeven, Utrecht (NL); Anika Kirsten Krohn, Utrecht (NL); Francisco Hernan Blanco Hedderich, Utrecht (NL)

(73) Assignee: N.V. Nutricia, Zoetermeer (NL)

(21) Appl. No.: 19/674,217

(22) Filed: May 12, 2026

Related U.S. Application Data
(62) Division of application No. PCT/EP2024/083121, filed on Nov. 21, 2024.

(30) Foreign Application Priority Data
Nov. 21, 2023 (EP) 23211299.5

(51) Int. Cl. A23L 33/00; A23L 33/115; A23L 33/125; A23L 33/135; A23L 33/19

(52) U.S. Cl. CPC A23L 33/40; A23L 33/115; A23L 33/125; A23L 33/135; A23L 33/19

(57) ABSTRACT

The invention relates to a particulate nutritional composition for infants with less powder segregation, wherein the particulate nutritional composition comprises milk formula particles and probiotic particles, which differ in particle size. The invention further relates to a process for producing the particulate nutritional composition and to a use of the particulate nutritional composition.

### FIELD OF THE INVENTION

The invention relates to a particulate nutritional composition for infants with less powder segregation, wherein the particulate nutritional composition comprises milk formula particles and probiotic particles, which differ in particle size. The invention further relates to a process for producing the particulate nutritional composition and to a use of the particulate nutritional composition.

### BACKGROUND OF THE INVENTION

Human milk is the uncontested gold standard concerning infant nutrition. However, in some cases breastfeeding is inadequate or unsuccessful for medical reasons or not available because of a choice not to breastfeed. For such situations infant or follow-on formulas have been developed. Commercial infant formulas are commonly used today to provide supplemental or the sole source of nutrition early in life. These formulas comprise a range of nutrients to meet the nutritional needs of the growing infant, and typically include fat, carbohydrate, protein, vitamins, minerals, and other nutrients helpful for optimal infant growth and development. Commercial infant formulas are designed to mimic, as closely as possible, the composition and function of human milk.

Human milk lipids are known to have a distinct physical structure composed of large lipid globules with a mode diameter, based on volume, of about 4 μm existing of a triglyceride core coated by a tri-layer of membranes, the milk fat globule membrane (MFGM). Standard infant formula's typically have lipid droplets with a mode diameter, based on volume, of about 0.3-0.5 μm due to industrial processing procedures applied to achieve stable products. The lipid droplets are not surrounded by MFGM, but mostly by milk proteins. Infant formula comprising lipid globules with an architecture more similar to the lipid globules in human milk have been described (e.g. WO2010027258 or WO2010/0027259).

Infant formula have to meet compositional requirements that are laid down in regulations such as the European Commission Directive 2006/141/EC of 22 Dec. 2006 on infant formulae and follow-on formulae.

Nutritional compositions for infants and young children are often sold as powders (i.e. as a particulate composition) to be reconstituted with water. A typical process for producing powdered infant milk formula comprises the following steps:
a) Providing an aqueous phase comprising the water soluble ingredients;
b) Providing a lipid phase;
c) Homogenizing the aqueous and lipid phase to provide an oil-in-water emulsion;
d) Spray drying the oil-in-water emulsion to provide spray-dried milk formula particles.

In addition, a heat treatment is applied before and/or after the homogenization step for pasteurization/sterilization. Both the heat treatment step and the spray-drying step expose the nutritional ingredients to heat. It is therefore common in the art to dry-blend the heat-sensitive ingredients with the spray-dried milk formula powder to obtain the final powdered infant formula.

Infant milk formula's may comprise probiotics. Probiotics are live beneficial microorganisms that are heat sensitive. Particles comprising probiotics are therefor usually dry-blended with the spray-dried infant formula particles.

The spray-dried milk formula particles and the particles comprising probiotics have different characteristics, which may result in powder segregation when transferring the powdered product between processing steps in the factory, during transport or storage of the packaged powdered composition. This powder segregation is undesired, as it is crucial for infant formula that each scope of powder provides the same amount of nutrients. There should be no difference between the powder in the top and the powder in the bottom of a packaging.

Powder segregation may be caused by the differences in size, shape or density of the powder particles. Particle size is probably the most significant source of segregation.

For example, when transferring a particulate composition between processing steps using pneumatic transport, the in-flight material will travel at different velocities depending on particle size, this creates what is known as trajectory segregation. Larger particles will be projected further, leaving the smaller ones behind.

A further cause of segregation is percolation. The small particles move downwards through the mass falling into the spaces between the larger particles. At the same time, the larger particles move upwards in the so called ‘Brazil Nut effect’ as voids are created then filled in with the smaller particles forcing the larger particles upwards. This type of segregation typically takes place after packaging of the powder, e.g. during transport of the packaged product on its journey from the factory to the consumer's home.

There remains a need for a particulate nutritional composition, comprising milk formula particles and probiotic particles, which shows reduced powder segregation.

### SUMMARY OF THE INVENTION

The inventors have unexpectedly found that milk formula particles comprising large, phospholipid coated lipid globules reduce the powder segregation in a particulate nutritional composition comprising both milk formula particles and probiotic particles, when the milk formula particles and probiotic particles differ in particle size.

Although the inventors do not wish to be bound by theory, it is believed that the large, phospholipid coated lipid globules are not homogeneously distributed within the spray-dried milk formula particles, but are more located on the outside of the spray-dried milk formula particles, which allows some form of interaction with the probiotic particles and thereby prevents powder segregation.

Accordingly, the present invention provides for a particulate nutritional composition selected from infant formula, follow-on formula, and young child formula, wherein the particulate nutritional composition comprises:
80-99.8 wt. % of milk formula particles comprising digestible carbohydrates, lipid, and protein, having a volume-weighted mode diameter of 50-500 μm;
0.1-10 wt. % of probiotic particles comprising probiotic bacteria and having a volume-weighted mode diameter of 1-100 μm;
   wherein the lipid in the milk formula particles is in the form of lipid globules, wherein
i. the lipid globules have a mode diameter based on volume of at least 1 μm and/or at least 45 vol. % of the lipid globules has a diameter of 2 to 12 μm, and
ii. the lipid comprises at least 0.5 wt. % phospholipid based on total lipid and the lipid globules have a coating comprising phospholipid.

In addition, the present invention provides a process to prepare such particulate nutritional composition, and a use of this particulate nutritional composition.

Finally, the present invention pertains to the use of milk formula particles to reduce powder segregation in a particulate nutritional composition comprising said milk formula particles and at least one other type of particles, wherein the milk formula particles comprise digestible carbohydrates, lipid, and protein, and wherein the lipid in the milk formula particles is in the form of lipid globules, wherein
i. the lipid globules have a mode diameter based on volume of at least 1 μm and/or at least 45 vol. % of the lipid globules has a diameter of 2 to 12 μm, and
ii. the lipid comprises at least 0.5 wt. % phospholipid based on total lipid and the lipid globules have a coating comprising phospholipid.

### DETAILED DESCRIPTION OF THE INVENTION

A first aspect of the invention relates to a particulate nutritional composition selected from infant formula, follow-on formula, and young child formula, wherein the particulate nutritional composition comprises:
80-99.8 wt. % of milk formula particles comprising digestible carbohydrates, lipid, and protein, having a volume-weighted mode diameter of 50-500 μm;
0.1-10 wt. % of probiotic particles comprising probiotic bacteria and having a volume-weighted mode diameter of 1-100 μm;
   wherein the lipid in the milk formula particles is in the form of lipid globules, wherein
i. the lipid globules have a mode diameter based on volume of at least 1 μm and/or at least 45 vol. % of the lipid globules has a diameter of 2 to 12 μm, and
ii. the lipid comprises at least 0.5 wt. % phospholipid based on total lipid and the lipid globules have a coating comprising phospholipid.

### Particulate Nutritional Composition

The probiotic particles comprise probiotic bacteria. Preferably, the probiotic bacteria are suitable for improving the gut microbiota in an human infant.

In a preferred embodiment, the probiotic bacteria are selected from Lactobacillus, Bifidobacterium and combinations thereof, more preferably the probiotic bacteria is Bifidobacterium, more preferably the probiotic bacteria is Bifidobacterium breve.

The particulate nutritional composition preferably has a dry matter content of at least 90 wt. %, more preferably 92.5-100 wt. %, even more preferably 95-99.9 wt. %.

Preferably, the particulate nutritional composition comprises 85-99.5 wt. % of milk formula particles, more preferably 90-99 wt. % of milk formula particles.

Preferably, the particulate nutritional composition comprises 0.2-7.5 wt. % of probiotic particles, more preferably 0.3-5 wt. % of probiotic particles.

The weight ratio between the milk formula particles and the probiotic particles in the particulate nutritional composition is preferably at least 10:1, more preferably 25:1-1500:1, and most preferably 50:1-1000:1.

The volume-weighted mode diameter of the particles relates to the diameter which is the most present based on volume of total particles, or the peak value in a graphic representation, having on the X-axis the diameter of the particles and on the Y-axis the volume (%). The volume of the particles and its size distribution can suitably be determined using a particle size analyzer such as a Mastersizer (Malvern Instruments, Malvern, UK).

The milk formula particles preferably have a volume-weighted mode diameter of 80-500 μm, more preferably a volume-weighted mode diameter of 80-400 μm, even more preferably a volume-weighted mode diameter of 120-300 μm, and most preferably a volume-weighted mode diameter of 150-250 μm.

Preferably, the probiotic particles have a volume-weighted mode diameter of 2-75 μm, more preferably a volume-weighted mode diameter of 4-50 μm, and most preferably 5-20 μm.

Preferably, the volume-weighted mode diameter of the milk formula particles is at least 2 times the volume-weighted mode diameter of the probiotic particles. More preferably, the volume-weighted mode diameter of the milk formula particles is at least 4 times the volume-weighted mode diameter of the probiotic particles. Most preferably, the volume-weighted mode diameter of the milk formula particles is 8-30 times the volume-weighted mode diameter of the probiotic particles.

The particulate nutritional composition has a low tendency to segregate. Segregation tendency of powers can be described by their segregation potential (SP), for example by using the method described in ASTM D6941-12. The lowest SP is, the lowest the tendency of a powder to segregate. Preferably the particulate nutritional composition has a segregation potential based on particle size distribution d₁₀ by volume (SP_{d10}) of less than 1.0%, more preferably less than 0.5 wt. %, most preferably about 0.3%, wherein SP_{d10} is measured according to ASTM D6941-12 as shown in the example. Preferably the particulate nutritional composition has a segregation potential based on particle size distribution d₅₀ by volume (SP 50) of less than 1.2%, more preferably less than 0.6 wt. %, most preferably about 0.4%, wherein SP_{d50} is measured according to ASTM D6941-12 as shown in the example. Preferably the particulate nutritional composition has a segregation potential based on particle size distribution deo by volume (SP_{d90}) of less than 1.5%, more preferably less than 0.8 wt. %, most preferably about 0.6% wherein SP_{d10} is measured according to ASTM D6941-12 as shown in the example.

In a preferred embodiment, the particulate nutritional composition is packaged in a container, said container comprising 100-1200 g of said particulate nutritional composition. In a more preferred embodiment, the invention pertains to a container comprising 100-1200 g of the particulate nutritional composition as described herein.

It is preferred that the particulate nutritional composition is suitable for dispersion into an aqueous liquid to provide a ready-to-drink liquid, preferably the aqueous liquid is selected from water, breast milk and combinations thereof, most preferably the aqueous liquid is water.

### Lipid Globule Size

The lipid is present in the milk formula particles in the form of lipid globules. The lipid globules comprise a core and a surface.

The lipid globules preferably have mode diameter, based on volume, of at least 1.0 μm, more preferably at least 3.0 μm, and most preferably at least 4.0 μm. Preferably, the lipid globules have a mode diameter, based on volume, between 1.0 and 10 μm, more preferably between 2.0 and 8.0 μm, even more preferably between 3.0 and 7.0 μm, and most preferably between 4.0 μm and 6.0 μm.

Alternatively, or preferably in addition, the size distribution of the lipid globules is preferably in such a way that at least 45 volume % (vol. %), preferably at least 55 vol. %, even more preferably at least 65 vol. %, and most preferably at least 75 vol. % of the lipid globules have a diameter between 2 and 12 μm. In a preferred embodiment, at least 45 vol. %, preferably at least 55 vol. %, more preferably at least 65 vol. %, and most preferably at least 75 vol. % of the lipid globules have a diameter between 2 and 10 μm. In a more preferred embodiment, at least 45 vol. %, more preferably at least 55 vol. %, yet even more preferably at least 65 vol. %, and most preferably at least 75 vol. % of the lipid globules have a diameter between 4 and 10 μm. Preferably less than 5 vol. % of the lipid globules have a diameter above 12 μm.

The percentage of lipid globules is based on volume of total lipid. The mode diameter relates to the diameter which is the most present based on volume of total lipid, or the peak value in a graphic representation, having on the X-axis the diameter and on the Y-axis the volume (%).

The volume of the lipid globules and its size distribution can suitably be determined using a particle size analyzer such as a Mastersizer 2000 (Malvern Instruments, Malvern, UK), for example by the method described in Michalski et al, 2001, Lait 81:787-796.

### Lipid

The milk formula particles comprise lipid. The term “lipid” as used herein comprises one or more selected from the group consisting of triglycerides, polar lipids (such as phospholipids, cholesterol, glycolipids, sphingomyelin), free fatty acids, monoglycerides and diglycerides.

The lipid provides preferably 30 to 60% of the total calories of the particulate nutritional composition. More preferably the lipid provides 35 to 55% of the total calories, even more preferably lipids provide 40 to 50% of the total calories. The lipid is preferably present in an amount of 4 to 6 g per 100 kcal. When in liquid form, e.g. as a ready-to-feed liquid, the particulate nutritional composition preferably comprises 2.1 to 6.5 g lipids per 100 ml, more preferably 3.0 to 4.0 g per 100 ml. Based on dry weight the particulate nutritional composition preferably comprises 10 to 50 wt. %, more preferably 12.5 to 40 wt. % lipids, even more preferably 19 to 30 wt. % lipids.

The lipid preferably comprises vegetable lipids. The presence of vegetable lipids advantageously enables an optimal fatty acid profile, high in polyunsaturated fatty acids and/or more reminiscent to human milk fat. Lipids from mammalian milk alone, e.g. cow's milk, do not provide an optimal fatty acid profile. The amount of essential fatty acids is too low in mammalian milk.

Preferably the lipid comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

In one preferred embodiment, the lipid comprises 5 to 100 wt. % vegetable lipids based on total lipids, more preferably 10 to 95 wt. %, more preferably 20 to 80 wt. %, even more preferably 25 to 75 wt. %, most preferably 40 to 60 wt. %. It is noted therefore that lipid also may preferably comprise non-vegetable lipids. Non-vegetable lipids may include mammalian milk fat, mammalian milk derived lipid as a preferred source of phospholipid, and fish, marine and/or microbial oils as source of LC-PUFA.

### Fatty Acid Composition

SFA relates to saturated fatty acids and/or acyl chains, MUFA relates to mono-unsaturated fatty acid and/or acyl chains, PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds; DHA refers to docosahexaenoic acid and/or acyl chain (22:6, n3); EPA refers to eicosapentaenoic acid and/or acyl chain (20:5 n3); ARA refers to arachidonic acid and/or acyl chain (20:4 n6); DPA refers to docosapentaenoic acid and/or acyl chain (22:5 n3). n3 or omega 3 PUFA refers to polyunsaturated fatty acids and/or acyl chains with 2 or more unsaturated bonds and with an unsaturated bond at the third carbon atom from the methyl end of the fatty acyl chain, n6 or omega 6 PUFA refers to polyunsaturated fatty acids and/or acyl chains with 2 or more unsaturated bonds and with an unsaturated bond at the sixth carbon atom from the methyl end of the fatty acyl chain.

The lipid preferably comprises LA, which refers to linoleic acid and/or acyl chain (18:2 n6). LA is an n6 PUFA and the precursor of n6 LC-PUFA and is an essential fatty acid as it cannot be synthesized by the human body. LA preferably is present in a sufficient amount in order to promote a healthy growth and development, yet in an amount as low as possible to prevent negative, competitive, effects on the formation of n3 PUFA and a too high n6/n3 ratio. The lipid therefore preferably comprises less than 25 wt. %, more preferably less than 20 wt. %, most preferably less than 15 wt. % LA based on total fatty acids. The lipid preferably comprises at least 5 wt. % LA based on fatty acids, preferably at least 7.5 wt. %, more preferably at least 10 wt. % based on total fatty acids.

The lipid preferably comprises ALA, which refers to alpha-linolenic acid and/or acyl chain (18:3 n3). ALA is a n3 PUFA and the precursor of n3 LC-PUFA and is an essential fatty acid as it cannot be synthesized by the human body. Preferably ALA is present in a sufficient amount to promote a healthy growth and development of the infant. The lipid therefore preferably comprises at least 0.5 wt. %, more preferably at least 1.0 wt. %, even more preferably at least 1.5 wt. %, and most preferably at least 2.0 wt. % ALA based on total fatty acids. Preferably the lipid comprises less than 10 wt. % ALA, more preferably less than 5.0 wt. % ALA based on total fatty acids.

The weight ratio LA/ALA preferably is well balanced in order to ensure an optimal n6/n3 PUFA, n6/n3 LC PUFA and DHA/ARA ratio in the cellular membranes. Therefore, the lipid preferably comprises a weight ratio of LA/ALA from 2 to 20, more preferably from 3 to 15, even more preferably from 5 to 12, most preferably from 5 to 10. Preferably the n6 PUFA/n3 PUFA weight ratio is from 3 to 20, more preferably from 3 to 15, even more preferably from 4 to 12, most preferably from 5 to 10.

Preferably, the lipid comprises n3 LC-PUFA, such as EPA, DPA and/or DHA, more preferably DHA. As the conversion of ALA to DHA may be less efficient in infants, preferably both ALA and DHA are present in the nutritional composition. Preferably the lipid comprises at least 0.05 wt. %, preferably at least 0.1 wt. %, more preferably at least 0.2 wt. %, of DHA based on total fatty acids. Preferably the lipid comprises not more than 2.0 wt. %, preferably not more than 1.0 wt. %, of DHA based on total fatty acids.

The lipid preferably comprises ARA. Preferably the lipid comprises at least 0.05 wt. %, more preferably at least 0.1 wt. %, most preferably at least 0.2 wt. % of ARA based on total fatty acids. As the group of n6 fatty acids, especially ARA counteracts the group of n3 fatty acids, especially DHA, the lipid preferably comprises relatively low amounts of ARA. Preferably the lipid comprises not more than 2.0 wt. %, preferably not more than 1.0 wt. % of ARA based on total fatty acids. Preferably the weight ratio between DHA and ARA is between ¼ to 4/1, more preferably between ½ to 2/1, more preferably between 0.6 and 1.5.

### Phospholipid

The lipid comprises at least 0.5 wt. % phospholipids based on total lipid. Preferably the lipid comprises 0.5 to 20 wt. %, more preferably 0.6 to 15 wt. %, even more preferably 0.7 to 10 wt. %, even more preferably 0.8 to 8 wt. %, most preferably 1 to 5 wt. % phospholipid based on total lipid.

The lipid globules have a coating comprising phospholipid. By ‘coating’ is meant that the outer surface layer of the lipid globules comprises phospholipid, whereas phospholipid is virtually absent in the core of the lipid globule. A suitable way to determine whether phospholipid is located on the surface of lipid globules is confocal laser scanning microscopy or transmission electron microscopy; see for instance Gallier et al. (A novel infant milk formula concept: Mimicking the human milk fat globule structure, Colloids and Surfaces B: Biointerfaces 136 (2015) 329-339).

Phospholipids are amphipathic of nature and include glycerophospholipids and sphingomyelin.

The lipid preferably comprises glycerophospholipids. Examples of glycerophospholipids are phosphatidylcholine (PC), phosphatidylserine (PS), phosphatidylethanolamine (PE), phosphatidylinositol (PI) and phosphatidylglycerol (PG). Preferably the lipid comprises one or more of PC, PS, PI and PE, more preferably the milk formula particles comprises at least PC.

The lipid preferably comprises sphingomyelin. Sphingomyelins have a phosphorylcholine or phosphorylethanolamine molecule esterified to the 1-hydroxy group of a ceramide. They are classified as phospholipid as well as sphingolipid, but are not classified as a glycerophospholipid nor as a glycosphingolipid. Preferably the lipid comprises 0.05 to 10 wt. % sphingomyelin based on total lipid, more preferably 0.1 to 5 wt. %, even more preferably 0.2 to 2 wt. %. Preferably the lipid comprises at least 5 wt. %, more preferably 5 to 40 wt. % sphingomyelin based on total phospholipid, more preferably 10 to 35 wt. %, even more preferably 15 to 35 wt. % sphingomyelin, based on total phospholipid.

The lipid preferably comprises glycosphingolipids. The term glycosphingolipids in the present context particularly refers to glycolipids with an amino alcohol sphingosine. The sphingosine backbone is O-linked to a charged head-group such as ethanolamine, serine or choline backbone. The backbone is also amide linked to a fatty acyl group. Glycosphingolipids are ceramides with one or more sugar residues joined in a beta-glycosidic linkage at the 1-hydroxyl position, and include gangliosides. Preferably the lipid contains gangliosides, more preferably at least one ganglioside selected from the group consisting of GM3 and GD3. Preferably the lipid comprises 0.1 to 10 wt. % glycosphingolipids based on total lipid, more preferably 0.5 to 5 wt. %, even more preferably 2 to 4 wt. % glycosphingolipids, based on total lipid.

The lipid preferably comprises cholesterol. The lipid preferably comprises at least 0.005 wt. % cholesterol based on total lipid, more preferably at least 0.02 wt. %, more preferably at least 0.05 wt. %, even more preferably at least 0.1 wt. % cholesterol based on total lipid. Preferably the amount of cholesterol does not exceed 10 wt. % based on total lipid, more preferably does not exceed 5 wt. %, even more preferably does not exceed 1 wt. % of cholesterol based on total lipid.

Preferred sources for providing the phospholipid, glycosphingolipid and/or cholesterol are egg lipids, milk fat, buttermilk fat and butter serum fat (such as beta serum fat). Another preferred source for phospholipid, particularly PC, is soy lecithin and/or sunflower lecithin.

The phospholipid is preferably derived from mammalian milk. The lipid preferably comprises phospholipid, glycosphingolipid and/or cholesterol from mammalian milk, preferably from mammalian milk of cows, mares, sheep, goats, buffalos, horses and camels. More preferably the lipid comprises phospholipid, glycosphingolipid and/or cholesterol from cow's milk.

Phospholipid derived from mammalian milk includes preferably phospholipid that is isolated from milk lipid, cream lipid, cream serum lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipid, glycosphingolipid and/or cholesterol is obtained from milk cream. Suitable commercially available sources for phospholipid from milk are BAEF, SM2, SM3 and SM4 powder of Corman, Salibra of Glanbia, Vivinal MFGM of FrieslandCampina and LacProdan MFGM-10 or PL20 from Arla.

The use of phospholipid from mammalian milk fat advantageously comprises the use of milk fat globule membranes, which are more reminiscent to the situation in human milk. The concomitant use of phospholipid derived from mammalian milk and triglycerides derived from vegetable lipids therefore enables the manufacture of coated lipid globules with a coating more similar to human milk, while at the same time providing an optimal fatty acid profile.

Preferably the phospholipid is derived from mammalian milk lipid, more preferably from milk fat globule membrane (MFGM). Preferably the phospholipid is derived from cow's milk lipid, more preferably from cow's MFGM.

Preferably the lipid comprises phospholipid and glycosphingolipid and more preferably the weight ratio of phospholipid:glycosphingolipid is from 2:1 to 12:1, more preferably from 2:1 to 10:1 and even more preferably 2:1 to 5:1.

Methods for obtaining lipid globules with an increased size and/or coating with phospholipid are for example described in WO 2010/0027258 and WO 2010/0027259.

### Digestible Carbohydrates

The milk formula particles comprise digestible carbohydrates. The digestible carbohydrates preferably provide 30 to 80% of the total calories of the particulate nutritional composition. Preferably the digestible carbohydrates provide 40 to 60% of the total calories of the particulate nutritional composition. Based on calories the particulate nutritional composition preferably comprises 5 to 20 g of digestible carbohydrates per 100 kcal, more preferably 7.5 to 15 g. When in liquid form, e.g. as a ready-to-feed liquid, the particulate nutritional composition preferably comprises 3 to 30 g digestible carbohydrate per 100 ml, more preferably 6 to 20, even more preferably 7 to 10 g per 100 ml. Based on dry weight the nutritional composition preferably comprises 20 to 80 wt. %, more preferably 40 to 65 wt. % digestible carbohydrates.

Preferred digestible carbohydrate sources are lactose, glucose, sucrose, fructose, galactose, maltose, starch and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the particulate nutritional composition preferably comprises at least 25 wt. % lactose, preferably at least 40 wt. %.

### Protein

The milk formula particles comprise protein. The protein preferably provides 5 to 15% of the total calories of the particulate nutritional composition, more preferably 6 to 12% of the total calories. More preferably protein is present in the particulate nutritional composition below 3.5 gram per 100 kcal, more preferably between 1.8 and 2.1 g protein per 100 kcal, even more preferably between 1.85 and 2.0 g protein per 100 kcal. The protein concentration in a nutritional composition is determined by the sum of protein, peptides and free amino acids. Based on dry weight, the particulate nutritional composition preferably comprises less than 12 wt. % protein, more preferably between 9.6 and 12 wt. %, even more preferably between 10 and 11 wt. % protein. Based on a ready-to-drink liquid product the particulate nutritional composition preferably comprises less than 1.5 g protein per 100 ml, more preferably between 1.2 and 1.5 g, even more preferably between 1.25 and 1.35 g protein per 100 ml.

The source of the protein should be selected in such a way that the minimum requirements for essential amino acid content are met and satisfactory growth is ensured. Hence protein sources based on cows' milk proteins such as whey, casein and mixtures thereof and proteins based on soy, potato or pea are preferred. In case whey proteins are used, the protein source is preferably based on acid whey or sweet whey, whey protein isolate or mixtures thereof. Preferably the nutritional composition comprises at least 3 wt. % casein based on dry weight. Preferably the casein is intact and/or non-hydrolyzed.

### Non Digestible Carbohydrates

In one embodiment, the particulate nutritional composition preferably comprises non-digestible oligosaccharides, more preferably the milk formula particles comprise the non-digestible oligosaccharides.

Preferably the particulate nutritional composition comprises non-digestible oligosaccharides with a degree of polymerization (DP) between 2 and 250, more preferably 3 and 60. The non-digestible oligosaccharides advantageously further reduce the relative abundance of opportunistic pathogens and improves the gut microbiota in human infants

Preferably the particulate nutritional composition comprises fructo-oligosaccharides, galacto-oligosaccharides and/or galacturonic acid oligosaccharides, more preferably fructo-oligosaccharides and/or galacto-oligosaccharides, even more preferably galacto-oligosaccharides, most preferably transgalacto-oligosaccharides. In a preferred embodiment the particulate nutritional composition comprises a mixture of galacto-oligosaccharides and fructo-oligosaccharides, more preferably transgalacto-oligosaccharides and fructo-oligosaccharides. Suitable non-digestible oligosaccharides are for example Vivinal®GOS (FrieslandCampina DOMO), Raftilin®HP or Raftilose® (Orafti).

Preferably, based on a ready-to-drink liquid product, the particulate nutritional composition comprises 80 mg to 2 g non-digestible oligosaccharides per 100 ml, more preferably 150 mg to 1.5 g, even more preferably 300 mg to 1 g per 100 ml. Based on dry weight, the particulate nutritional composition preferably comprises 0.25 wt. % to 20 wt. %, more preferably 0.5 wt. % to 10 wt. %, even more preferably 1.5 wt. % to 7.5 wt. % of non-digestible oligosaccharides.

### Formula

In the present context, infant formula refers to nutritional compositions, artificially made, intended for infants of 0 to about 4 to 6 months of age and are intended as a substitute for human milk. Typically infant formulae are suitable to be used as sole source of nutrition. Such formulae are also known as starter formula. Formula for infants starting with at 4 to 6 months of life to 12 months of life are intended to be supplementary feedings to infants that start weaning on other foods. Such formulae are also known as follow-on formulae. Infant formulae and follow-on formulae are subject to strict regulations, for example for the EU Commission Directive 2006/141/EC. In the present context, young child formulae refers to nutritional compositions, artificially made, intended for infants of 12 months to 36 months, which are intended to be supplementary feedings to infants. Such formulae are also known as growing-up milks.

The particulate nutritional composition is preferably an infant formula or a follow-on formula. More preferably the particulate nutritional composition is an infant formula.

The particulate nutritional composition is preferably an infant formula or follow-on formula and preferably comprises 3 to 7 g lipid/100 kcal, more preferably 4 to 6 g lipid/100 kcal, moat preferably 4.5 to 5.5 g lipid/100 kcal, preferably comprises 1.7 to 5 g protein/100 kcal, more preferably 1.8 to 3.5 g protein/100 kcal, even more preferably 1.8 to 2.1 g protein/100 kcal, most preferably 1.8 to 2.0 g protein/100 kcal and preferably comprises 5 to 20 g digestible carbohydrate/100 kcal, more preferably 6 to 16 g digestible carbohydrate/100 kcal, and most preferably 10 to 15 g digestible carbohydrate/100 kcal.

Preferably the particulate nutritional composition is an infant formula or follow-on formula, and, when in a ready-to-drink format, has an energy density of 60 kcal to 75 kcal/100 ml, more preferably 60 to 70 kcal/100 ml. This density ensures an optimal balance between hydration and caloric intake.

### Use

A second aspect of the invention pertains to the use of the particulate nutritional composition according to the invention for improving the gut microbiota in a human infant.

For some jurisdictions, the invention may also be worded as a method for improving the gut microbiota in a human infant, said method comprising the administration of the particulate nutritional composition according to the invention.

For some jurisdictions, the invention may also be worded as the use of the milk formula particles and probiotic particles for the manufacture of the particulate nutritional composition according to the invention for improving the gut microbiota in a human infant, wherein the nutritional composition is selected from infant formula, follow-on formula, and young child formula.

In some jurisdictions administering a nutritional composition to an infant is considered non-therapeutic. In those instances the invention may be worded as defined above by way of a method comprising administering a nutritional composition. For clarity, the method can also be defined as a non-therapeutic method. By definition, the words “non-therapeutic” exclude any therapeutic effect.

“Improving the gut microbiota in a human infant” as used herein, preferably refers to preventing or treating a compromised gut microbiota in a human infant.

In a preferred embodiment, the human infant is at risk of having compromised gut microbiota. Preferably, the human infant at risk of having compromised gut microbiota is selected from the group of infants born via caesarean section, infants born from a mother who received antibiotics, infants receiving or having received antibiotics, infants receiving or having received proton pump inhibitors, or combinations thereof. More preferably, the human infant at risk of having a compromised gut microbiota is selected from the group of infants born via caesarean section, infants born from a mother who received antibiotics, or combinations thereof. Most preferably, the human infant at risk of having a compromised gut microbiota is an infant born via caesarean section.

“Infants born from a mother who received antibiotics” as used herein refers to infants born from a mother who received antibiotics in the two weeks preceding the delivery or during the delivery (intrapartum). Preferably, “infants born from a mother who received antibiotics” refers to infants born from a mother who received intrapartum antibiotics.

Preferably, the human infant is aged 0-36 months, more preferably 0-24 months, even more preferably 0-12 months and most preferably 0-6 months.

Preferably, all embodiments described herein above in the context of the first aspect of the invention equally apply to this second aspect of the invention.

### Process

A third aspect of the invention relates to a process for producing the particulate nutritional composition according to the invention, said process comprises the steps of:
a. providing an aqueous phase with a dry matter content of 10-60 wt. %, based on total weight of the aqueous phase, which comprises at least one protein component,
b. providing a liquid lipid phase, which comprises at least one lipid;
c. mixing the lipid phase with the aqueous phase to provide an oil-in-water emulsion;
d. drying the oil-in-water emulsion to provide the milk formula particles;
e. providing probiotic particles comprising probiotic bacteria; and
f. dry-blending the milk formula particles and the probiotic particles to provide the particulate nutritional composition.

WO2010/027259, WO2013/135739, WO2013/135738 and WO2016/146496 disclose examples of the above process steps a. to d. to prepare the milk formula particles with the larger phospholipid coated lipid globules. Preferably the drying step d. encompasses spray-drying.

In a preferred embodiment, the particulate nutritional composition is obtainable by the process described above.

Preferably, all embodiments described herein above in the context of the first aspect of the invention equally apply to this third aspect of the invention.

Preferably, the milk formula particles and the probiotic particles are dry-blended in a weight ratio of at least 10:1, more preferably 25:1-1500:1, and most preferably 50:1-1000:1.

Preferably, after the dry-blending step f., the particulate nutritional composition is transported to a packaging unit, wherein the transport is done by pneumatic transport, gravity transport, or combinations thereof. More preferably, the particulate nutritional composition is transported to the packaging unit by gravity transport.

Preferably, the provided particulate nutritional composition in step f. is packaged in a container in the packaging unit, wherein the weight of the particulate nutritional composition in the container is in the range of 100-1200 g.

### Use

A fourth aspect of the invention relates to the use of milk formula particles to reduce powder segregation in a particulate nutritional composition comprising said milk formula particles and at least one other type of particles, wherein the milk formula particles comprise digestible carbohydrates, lipid, and protein, and Wherein the lipid in the milk formula particles is in the form of lipid globules, wherein
i. the lipid globules have a mode diameter based on volume of at least 1 μm and/or at least 45 vol. % of the lipid globules has a diameter of 2 to 12 μm, and
ii. the lipid comprises at least 0.5 wt. % phospholipid based on total lipid and the lipid globules have a coating comprising phospholipid.

Preferably, the milk formula particles and the at least one other type of particles differ in size, more preferably they differ in their volume-weighted mode diameter.

In a preferred embodiment, the volume-weighted mode diameter of the milk formula particles is at least 2 times the volume-weighted mode diameter of the other type of particles. More preferably, the volume-weighted mode diameter of the milk formula particles is at least 4 times the volume-weighted mode diameter of the other type of particles. Most preferably, the volume-weighted mode diameter of the milk formula particles is at least 8-30 times the volume-weighted mode diameter of the other type of particles.

The milk formula particles preferably have a volume-weighted mode diameter of 50-500 μm, more preferably a volume-weighted mode diameter of 80-400 μm, even more preferably a volume-weighted mode diameter of 120-300 μm and most preferably 150-250 μm.

Preferably, the other type of particles have a volume-weighted mode diameter of 1-100 μm, more preferably 2-75 μm, even more preferably 4-50 μm and most preferably 5-20 μm.

The other type of particles preferably comprise one or more ingredients selected from probiotic bacteria, heat-sensitive nutrients such as vitamins, non-digestible oligosaccharides, sources of LCPUFA's, enzymes, or combinations thereof.

The other type or particles preferably encompasses one or more different types of particles. These different types of particles are different from each other in size, shape, weight, or content.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

### EXAMPLES

### Infant Milk Formula Powders

Two different infant milk formula's (IMF) were prepared according to the recipe in Table 1. The powders were blended in TURBULA® T2G/T2GE 3D shaker mixer for 10 minutes at 23 rpm.

Both IMF's comprised levels of lactose, milk protein, lipids, vitamins, minerals and nutrients in line with infant formula regulations. The majority of these ingredients were comprised in the spray-dried milk powder particles. The probiotic particles comprised Bifidobacterium breve (B. breve M-16V, Morinaga) as probiotic. The remainder in Table 1 was comprised of heat-sensitive nutrients which were dry-blended with the spray-dried milk powder particles.

The milk powder particles in IMF A were prepared according to a standard process for producing infant milk formula and therefore comprised lipid globules with a lower volume-weighted mode diameter. The milk powder particles in IMF 1 were prepared according to a process for preparing infant milk formula with a larger lipid globule size and a phospholipid coating surrounding the lipid globules, such as described in WO2010/027259 or WO2016146496. The lipid in the milk powder particles of IMF 1 comprised about 1.5 wt. % milk-derived phospholipids by weight of total lipid, whereas IMF A comprised no milk-derived phospholipids.

The particle size distribution of the powder particles and the lipid globules were determined using a Mastersizer 3000. Before determining the lipid globule size, the IMF's were first reconstituted to a ready-to-feed liquid.

TABLE 1

| | | IMF 1 | IMF A |
|---|---|---|---|
| Milk powder | Wt. % of total IMF | 97.75 | 97.4 |
| particles | Volume-weighted mode diameter (μm) | ~200 | ~200 |
| | of particles | | |
| | Volume-weighted mode diameter (μm) | 3-5 | 0.3-0.5 |
| | of lipid globules within the particles | | |
| | Wt. % milk phospholipids by weight of | 1.5 | 0 |
| | total lipid | | |
| Probiotic | Wt. % of total IMF | 1 | 1 |
| particles | Volume-weighted mode diameter (μm) | 15 | 15 |
| | of particles | | |
| Remainder | Wt. % of total IMF | 1.25 | 1.6 |

### Segregation Assessment

For the physical analysis, the segregation risk of the different blends was assessed. The Jenike & Johanson segregation tester was used to assess segregation using the method described in ASTM D6941-12. The goal of the fluidization segregation test is to bring the material in the test chamber to a completely fluidized/aerated state, then to allow slow deaeration (settlement) of the powder. When the test is finished and all powder in the chamber is settled, samples can be collected. The column of powder was split in three sections and each section (top, middle, bottom) was analyzed for segregation.

Both the top and the bottom sample as well as the original sample (prior to being fluidized) were characterized in terms of particle size distribution (PSD) by means of the Malvern Mastersizer 3000. From the PSD, three characteristic parameters: d₁₀, d₅₀ and deo, all by volume, were recorded for each of the samples. The segregation potential (SP) is then calculated as:

$$SP = \vert \frac{{d}_{top} - {d}_{bottom}}{{d}_{0}} \vert \cdot 100 \%$$

Where d is any of the three quantiles (d₁₀, d₅₀ or d₉₀, all by volume). The sub-index bottom and top refer to the separation done after fluidization, while do refers to the quantile value prior to the powder being admitted to the test chamber. A lower value for SP indicated less segregation potential.

The results of the test are shown in Table 2. From the table it can be derived that less segregation occurs with the product of the invention, indicated by the lower absolute SP values compared to the control product.

TABLE 2

| | SP_{d10} | SP_{d50} | SP_{d90} |
|---|---|---|---|
| IMF 1 | 0.3% | 0.4% | 0.6% |
| IMF A | 4.0% | 9.7% | 10.9% |

1. A particulate nutritional composition selected from infant formula, follow-on formula, and young child formula, wherein the particulate nutritional composition comprises:
80-99.8 wt. % of milk formula particles comprising digestible carbohydrates, lipid, and protein, having a volume-weighted mode diameter of 80-500 μm;
0.1-10 wt. % of probiotic particles comprising probiotic bacteria and having a volume-weighted mode diameter of 2-75 μm;
wherein the lipid in the milk formula particles is in the form of lipid globules, wherein
i. the lipid globules have a mode diameter based on volume of at least 1 μm and/or at least 45 vol. % of the lipid globules has a diameter of 2 to 12 μm, and
ii. the lipid comprises at least 0.5 wt. % phospholipid based on total lipid and the lipid globules have a coating comprising phospholipid.

2. The particulate nutritional composition according to claim 1, wherein the weight ratio between the milk formula particles and the probiotic particles is at least 10:1.

3. The particulate nutritional composition according to claim 1, wherein the volume-weighted mode diameter of the milk formula particles is at least 2 times the volume-weighted mode diameter of the probiotic particles.

4. The particulate nutritional composition according to claim 1, wherein the milk formula particles have a volume-weighted mode diameter of 150-250 μm.

5. The particulate nutritional composition according to claim 1, wherein the probiotic particles have a volume-weighted mode diameter of 5-20 μm.

6. The particulate nutritional composition according to claim 1, wherein the dry matter content of the particulate nutritional composition is at least 90 wt. %.

7. The particulate nutritional composition according to claim 1, wherein the probiotic bacteria are selected from Lactobacillus, Bifidobacterium and combinations thereof.

8. The particulate nutritional composition according to claim 1, wherein the phospholipids are mammalian milk-derived phospholipids, preferably the phospholipids are derived from milk fat globule membrane (MFGM).

9. The particulate nutritional composition according to claim 1, wherein the particulate nutritional composition is packaged in a container, said container comprising 100-1200 g of said particulate nutritional composition.

10.-11. (canceled)

12. A process for producing the particulate nutritional composition according to claim 1, wherein the process comprises the steps of:
a. providing an aqueous phase with a dry matter content of 10-60 wt. %, based on total weight of the aqueous phase, which comprises at least one protein component,
b. providing a liquid lipid phase, which comprises at least one lipid;
c. mixing the lipid phase with the aqueous phase to provide an oil-in-water emulsion;
d. drying the oil-in-water emulsion to provide the milk formula particles;
e. providing probiotic particles comprising probiotic bacteria; and
f. dry-blending the milk formula particles and the probiotic particles to provide the particulate nutritional composition.

13. The process according to claim 12, wherein the drying step d. encompasses spray-drying.

14.-15. (canceled)

16. A method for improving the gut microbiota in a human infant, the method comprising administering the particulate nutritional composition according to claim 1 to the infant.

17. The method according to claim 16, wherein the human infant is at risk of having a compromised gut microbiota, preferably the human infant is selected from the group of infants born via caesarean section, infants born from a mother who received antibiotics, infants receiving or having received antibiotics, infants receiving or having received proton pump inhibitors, or combinations thereof.
